(19) United States
(12) Patent Application Publication
(10) Pub. No.: US 2021/0322700 A1
(43) Pub. Date: Oct. 21, 2021

(54) TUBE CHANGER DEVICE AND METHODS OF USE THEREOF

(71) Applicant: University of Florida Research Foundation, Inc., Gainesville, FL (US)

(72) Inventors: Nikolaus Gravenstein, Gainesville, FL (US); Tomas D. Martin, Orlando, FL (US); John Everett Martin, Gainesville, FL (US)

(21) Appl. No.: 17/232,768

(22) Filed: Apr. 16, 2021

Related U.S. Application Data
(60) Provisional application No. 63/010,835, filed on Apr. 16, 2020.

(51) Int. Cl. A61M 16/04

(52) U.S. Cl. CPC A61M 16/0488; A61M 2025/0004

(57) ABSTRACT

Various embodiments of a tube changer device and methods of use are described herein. A tube changer device to aid in placement or replacement of an endotracheal tube including tubular shaft having a hollow interior forming a main lumen extending from a proximal end to a distal end and a dilator portion at the distal end. The dilator portion having a tapered profile with an outer diameter that varies along a length measured from a junction point on the tubular shaft to a blunt tip. The main lumen extending through the dilator portion to the blunt tip. The tube changer device configured to be slidably received within the endotracheal tube. The tubular shaft can be flexible. The tube changer device can be used with or without a stylet for the placement or replacement of an endotracheal tube.

### CROSS REFERENCE TO RELATED APPLICATIONS

This application claims the benefit of U.S. Provisional Patent Application No. 63/010,835, filed Apr. 16, 2020, the entire contents of which is hereby incorporated herein by reference.

### BACKGROUND

Endotracheal tubes are commonly used for airway management in the settings of general anesthesia, critical care, mechanical ventilation, and emergency medicine. An endotracheal tube is a catheter that is inserted into the trachea for the primary purpose of establishing and maintaining a patent airway and to ensure the adequate exchange of oxygen and carbon dioxide. Under certain conditions, an endotracheal tube may need to be replaced or exchanged for continued ventilation of a patient.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 illustrates an example of a tube changer device according to various embodiments described herein.

FIG. 2 illustrates an example a tube changer device shown threaded onto a stylet and positioned within an endotracheal tube according to various embodiments described herein.

FIG. 3 illustrates an example method for replacing an endotracheal tube using the tube changer device of FIG. 1 according to various embodiments described herein.

FIG. 4 illustrates an example method for placing an endotracheal tube using the tube changer device of FIG. 1 according to various embodiments described herein.

The drawings illustrate only example embodiments and are therefore not to be considered limiting of the scope of the embodiments described herein, as other embodiments are within the scope of this disclosure. The elements and features shown in the drawings are not necessarily drawn to scale, emphasis instead being placed upon clearly illustrating the principles of the embodiments. Additionally, certain dimensions or positionings may be exaggerated to help visually convey certain principles. In the drawings, similar reference numerals between figures designate like or corresponding, but not necessarily the same, elements.

### DETAILED DESCRIPTION

An endotracheal tube (ETT) is a particular type of catheter that is used for airway management in the settings of general anesthesia, critical care, mechanical ventilation, and emergency medicine to establish and maintain a patent airway and to ensure the adequate exchange of oxygen and carbon dioxide. A medical procedure commonly known as intubation involves inserting the ETT (also referred to herein as a tube, catheter, and/or intubation catheter) via a patient's oral or nasal passage to provide a passageway to a lung or lungs for ventilation. Once a patient has been intubated, it may be necessary to replace or exchange the catheter due to improper placement, cuff malfunction, or to change the size or type of ETT. For example, an endobronchial tube used during surgery may need to be exchanged for an endotracheal tube when there is expected to be prolonged ventilation of the patient after surgery.

Most endotracheal tubes have a bevel tip and an opening in a side wall near the tip. Complications can occur during placement of an endotracheal tube such as lacerations, damage to the vocal cords, bleeding, injury to the throat or trachea, or other trauma to the upper airway. This can further complicate the care and recovery of the patient. Replacement of an existing ETT can be particularly difficult due to edema or swelling. Once an ETT is removed, there is a possibility that the airway may close. In such a case, there is a need to keep the airway passage open to replace the tube without causing trauma to the pharynx, larynx, or vocal cords.

When the visualization of the vocal cords is difficult, a stylet or bougie can be inserted into the airway and used as a guide to advance an ETT though the passage to proper placement. A stylet or bougie, such as an Eschmann stylet, is a coated, malleable, elongated cylinder and relatively smaller in diameter as compared to an ETT, which can facilitate passage through the airway. Conventional stylets can be rigid and/or be configured with an angled tip or bend in the stylet. While a stylet or bougie can be used for an initial placement of an ETT, it can also be used as a guide for the replacement of an ETT. For replacement of a tube, the stylet or bougie can be inserted through an existing ETT positioned in the patient's airway, after which the ETT is withdrawn with the stylet or bougie left in place. The replacement ETT is guided over the stylet past the arytenoids and vocal cords and positioned. The stylet or bougie is then removed for the patient to be ventilated through the replaced ETT.

In the context outlined above, the embodiments described herein are directed to a tube changer device configured to aid in the placement or replacement of an endotracheal tube in a patient. The tube changer device is hollow and flexible with a tapered distal end configured to minimize trauma during placement of the ETT. When inserted in an endotracheal tube, the tapered distal dilator portion of the tube changer device is configured to cover the distal wall opening of the endotracheal tube and the blunt tip of the dilator portion extends to or past the bevel tip of the endotracheal tube to provide a blunted end as the endotracheal tube is placed. The tube changer device provides an alternative to conventional stylets used for tube exchanges, including an Eschmann stylet and the like. In some embodiments, the tube changer device can be used to place or replace an ETT. In some embodiments, the tube changer device can be used in conjunction with a stylet to place or replace an ETT. When being used as guide as an alternative or in conjunction with a stylet, the tapered distal dilator portion of the tube changer device can also push away or through a constricted passage at a critical location and a limited airway during the procedure.

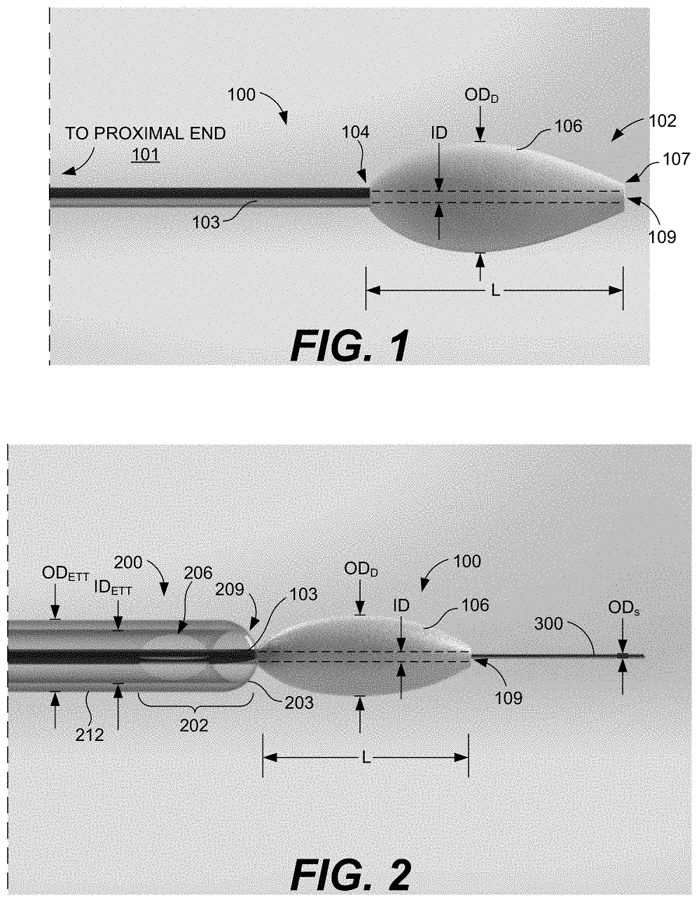

FIG. 1 illustrates an example of a tube changer device 100. The tube changer device 100, having a proximal end 101 and distal end 102, includes a flexible tubular body 103 and a dilator portion 106 forming a blunt tip 107 at the distal end 102 of the tube changer device 100. The flexible tubular body 103 is hollow and the dilator portion 106 can be formed in or secured to the flexible tubular body 103, such that a main lumen 109 extends from the proximal end 101 of the tube changer device 100 to an open distal end 102 at the blunt tip 107 of the dilator portion 106. The flexible tubular body 103 can be formed from polyurethane, polyvinyl chloride, or similar biocompatible materials. The dilator portion 106 can have a tapered profile with an outer diameter (OD_{D}) that varies along a length (L) measured from a junction point 104 on the flexible tubular body 103 to the blunt tip 107 at the distal end 102. The main lumen 109 has an interior diameter (ID). The dilator portion 106 can have a maximum diameter between the junction point and the distal end 102 forming a tapered profile.

FIG. 2 illustrates the tube changer device 100 of FIG. 1 inserted in an intubation catheter 200 and threaded over a stylet 300. The tube changer device 100 can be used with or without a stylet 300. As shown, an example of an intubation catheter 200 has a distal portion 202 that includes a bevel tip 203 and a distal wall opening 206 on a side wall 212 of the intubation catheter 200. The intubation catheter 200 has an inner diameter (ID_{ETT}) and outer diameter (OD_{ETT}) forming a single lumen 209. In this illustration, the tube changer device 100 is positioned with the dilator portion 106 extending beyond the bevel tip 203 of the intubation catheter 200. The dilator portion 106 of the tube changer device 100 can be configured to fit within the intubation catheter 200 to blunt the edges of the bevel open tip 203 to minimize trauma to the patient as the tube 200 is being placed. The dilator portion 106 can also blunt edges of the distal wall opening 206. The distal end 102 of the tube changer device 100 can be configured to be slidably received within an intubation catheter 200. The maximum diameter of the dilator portion 106 can be about equal to an inner diameter of the intubation catheter, wherein the dilator portion 106 can slide within the lumen 209 of the intubation catheter 200. The maximum diameter of the dilator portion 106 can be greater than an inner diameter of the intubation catheter and configured to compress or deflate to slide within the intubation catheter. In an example, the dilator portion 106 can deform such that it can slide within the lumen 209 of the intubation catheter 200, then fill the space to blunt the bevel tip 203 and a distal wall opening 206.

In some embodiments, the dilator portion 106 comprises a solid tapered portion configured to provide a blunt tip 107. In some embodiments, the dilator portion 106 can have a substantially prolate spheroid or ovoid shape. The dilator portion 106 can be formed from material firm enough to push away or through a constricted passage, yet soft enough to protect the tissues of the airway. For example, the dilator portion 106 can be formed from polyurethane, polyvinyl chloride, or similar biocompatible materials. In some embodiments, the dilator portion 106 can be made of a sponge-like compressible material that enlarges circumferentially into the available space to a predetermined size limit. In some embodiments, the dilator portion 106 comprises an inflatable portion that can be inflated by a syringe via an inflation lumen.

The device can be sized and configured with measurements to correspond with a range of sizes of endotracheal tubes to be placed. When inserted in the intubation catheter 200 to the distal portion, the dilator portion of the device is configured to cover the distal wall opening of the intubation catheter and the blunt tip of the dilator portion extends to or past the bevel tip of the intubation catheter.

For example, the device can be configured to fit within an intubation catheter or ETT having an internal diameter ranging from about 2.0 mm to about 10.5 mm. As such, the dilator portion 106 of the tube changer device 100 can be sized to slidably fit within the inner diameter of a specified ETT, with a maximum outer diameter ranging from about 2.0 mm to about 10.5 mm. The length of the tube changer device 100 can be substantially longer than a standard ETT to allow the extraction of the tube changer device 100 after placement of the ETT. The length of the tube changer device 110 can range from about 50 cm to about 70 cm, depending on the configuration.

In an example, the tube changer device 100 can be used with both an intubation catheter 200 and a stylet 300. The stylet 300 can be a conventional stylet or bougie, such as an Eschmann stylet. In some embodiments, the stylet 300 can be configured to slide within the lumen 109 of the tube changer 100. As shown in FIG. 2, the lumen 109 of the tube changer device 100 can be configured with an inner diameter (ID_{D}) that can slidably receive an outer diameter (OD_{S}) the stylet 300. The main lumen 109 can be sized to correspond with the outer diameter of the stylet 300. The tube changer device 100 and the intubation catheter 200 can be guided as a unit over the stylet 300 that has been placed in the patient's airway. Alternately, the stylet 300 can be used as a guide for placement of the tube changer device 100, then the tube changer device 100 used as a guide for placement of the intubation catheter 200.

The tube changer 100 can be used with or without a stylet 300. The tube changer device 100 can be used in place of a stylet or bougie for ETT placement or replacement. The tube changer device 100 can be used in conjunction with a stylet or bougie to aid in opening constricted passages. The tube changer device 100 can be inserted in an ETT to blunt the edges of the distal wall opening 206 and open bevel tip 203 of an ETT to minimize trauma as the tube changer device 100 and ETT 200, together, are advanced over a stylet 300.

For example, the tube changer 100 can be used with only an intubation catheter 200. The tube changer device 100 can be configured to be used in place of a stylet or bougie. The tube changer device 100 can be configured to have a dilator portion 106 with a diameter corresponding to the inner diameter of the replacement tube. The dilator portion 106 of the tube changer device 100 can be guided through an existing ETT to provide a guided passage for a replacement ETT. In this case, the dilator portion 106 can be placed to hold open an airway for a patient at risk of life-threatening loss of airway. While the patient may still be at risk of trauma from the bevel tip of the ETT being guided through a swollen or compromised passage, the larger diameter dilator portion 106 can resist constriction at a critical location and the main lumen 103 can maintain a limited airway during the procedure. When the replacement ETT is advanced to the position of the dilator portion 106, the bevel tip of the ETT is gently guided over the tapered end of the tube changer device 100 and into position. The tube changer device 100 can then be removed from the replacement tube.

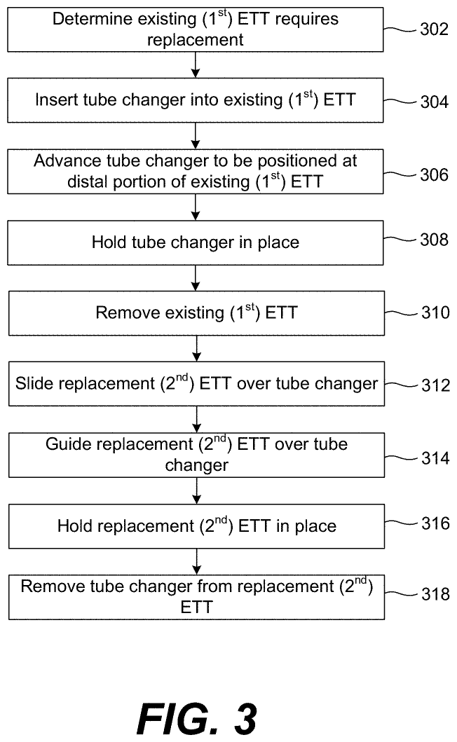

FIG. 3 shows an example method for placement of an ETT or intubation catheter. At step 302, determine that an existing ETT or first intubation catheter placed in an airway of a patient requires replacement. The first intubation catheter having a lumen extending from a proximal end exterior to the patient to a distal end placed within the patient. At step 304, inserting a distal end of a tube changer device 100 into the proximal end of the lumen of the first intubation catheter. At step 306, the tube changer device 100 is advanced through lumen of the first intubation catheter until the blunt tip 107 of the dilator portion 106 of the tube changer device 100 is positioned substantially aligned with the distal portion 202 of the first intubation catheter. At step 308, the tube changer device 100 is held in place. At step 310, remove the first intubation catheter by sliding the first intubation catheter over the tube changer device 100. At step 312, the second intubation catheter is introduced by placing a distal end of a second intubation catheter around the proximal end of the tube changer device 100. At step 314, advance the second intubation catheter guided by the tube changer device 100 until a distal portion of the second intubation catheter surrounds the dilator portion 106 of the tube changer device 100. At step 316, the second intubation catheter is held in place. At step 318, remove the tube changer device 100 from the second intubation catheter.

This method can be modified to be use the tube changer device 100 in conjunction with a conventional stylet. For example, before inserting the tube changer at step 304, a stylet 300 can be advanced into the existing ETT using a traditional method. The stylet 300 can be used as a guide. Step 304 of inserting a distal end of the tube changer device into the proximal end of the lumen of the first intubation catheter can include threading the tube changer device 100 over the stylet 300. Step 318 of removing the tube changer device from the second intubation catheter can include removing the stylet 300 together with the tube changer device 100.

In another example, after determining that a first intubation catheter placed in an airway of a patient requires replacement, a stylet 300 can be inserted into the first intubation catheter and advanced to a position. While holding the stylet 300 in position, the first intubation catheter can be removed by sliding the first intubation catheter over the stylet 300. Next, by inserting a distal end 102 of the tube changer device 100 into a lumen 209 of a second intubation catheter until a blunt portion 107 of the dilator portion 106 of the tube changer device 100 is positioned substantially aligned with the distal end 202 of the second intubation catheter. Together, the tube changer device 100 within the second intubation catheter can be positioned around a proximal end of the stylet 300, and advanced together. The tube changer device 100 and the second intubation catheter can be guided by the stylet 300 until the blunt portion 107 of the tube changer device 100 reaches a position. While holding the second intubation catheter 200b in position, the tube changer device 100 and stylet 300 can be removed, together, by sliding the tube changer device 100 through the lumen of the second catheter.

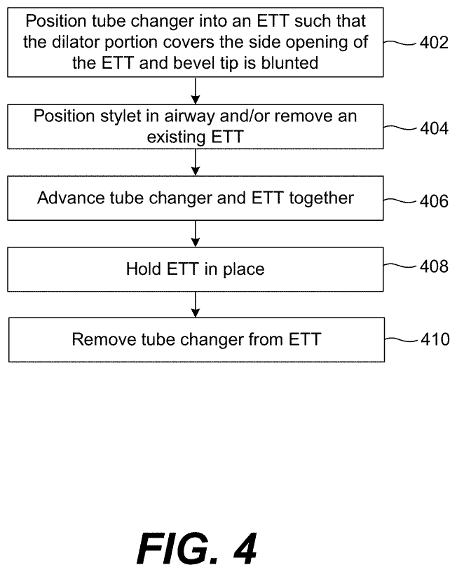

FIG. 4 shows an example method for placement of an ETT or intubation catheter where the tube changer device 100 and ETT 200 are advanced together. At step 402, position the tube changer 100 into an ETT 200 such that the dilator portion 106 covers the side opening 206 of the ETT 200 and bevel tip 203 is blunted by the blunt tip 107 of the dilator portion 106. The tube changer device 100 is slidably received within a lumen of the ETT 200. The ETT 200 can be replacement intubation catheter. When used with a stylet as a guide, at step 404, the stylet 300 can be positioned in the airway. In the case of a tube exchange, where an existing ETT is replaced, the stylet 300 can be inserted into an existing ETT and held in place and the existing ETT is removed. At step 406, the tube changer device 100 and the ETT 200 are advanced, together, until ETT 200 is in position and properly placed. When used with a stylet as a guide, the tube changer device 100 is threaded over the stylet 300 and tube changer device 100 and the ETT 200 are advanced, together, until ETT 200 is in position and properly placed. At step 406, the ETT is held in place. At step 408, the tube changer device 100 is removed from the ETT. When a stylet 300 is used, the tube changer device 100 and stylet 300 are removed, together, from the ETT.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation so as to encompass modifications and equivalent structures.

At least the following is claimed:

1. A tube changer device to aid in placement or replacement of an intubation catheter, the device comprising:
a tubular shaft having a hollow interior forming a main lumen extending from a proximal end to a distal end; and
a dilator portion at the distal end, the dilator portion having a tapered profile with an outer diameter that varies along a dilator length of the dilator portion, the dilator length being measured from a junction point of the dilator portion on the tubular shaft to a blunt tip of the dilator portion, the main lumen extending through the dilator portion to the blunt tip,
wherein the device is configured to be slidably received within the intubation catheter.

2. The tube changer device of claim 1, wherein the dilator length of the dilator portion of the device is greater than a distal portion of the intubation catheter, the distal portion of the intubation catheter comprising a bevel tip and a distal wall opening of the intubation catheter.

3. The tube changer device of claim 2, wherein, when inserted in the intubation catheter to the distal portion of the intubation catheter, the dilator portion is configured to cover the distal wall opening of the intubation catheter with the blunt tip of the dilator portion extending to or past the bevel tip of the intubation catheter.

4. The tube changer device of claim 1, wherein the dilator portion has a prolate spheroid or an ovoid shape.

5. The tube changer device of claim 1, wherein the outer diameter of the dilator portion has a maximum diameter configured to fit an inner diameter of the intubation catheter.

6. The tube changer device of claim 1, wherein the dilator portion has a maximum outer diameter ranging from about 2.0 mm to about 10.5 mm.

7. The tube changer device of claim 1, wherein the main lumen has an inner diameter configured to slidably receive a stylet.

8. The tube changer device of claim 1, wherein the tubular shaft is flexible.

9. The tube changer device of claim 1, wherein the dilator portion is formed in one piece with the tubular shaft.

10. The tube changer device of claim 1, wherein the dilator portion is secured to the tubular shaft.

11. The tube changer device of claim 1, wherein the dilator portion comprises polyurethane or polyvinyl chloride material.

12. The tube changer device of claim 1, wherein the dilator portion is inflatable.

13. The tube changer device of claim 1, further comprising an inflation lumen within a sidewall of the tubular shaft, the inflation lumen configured to deliver air between an inflation port at the proximal end and the dilator portion to inflate or deflate the dilator portion.

14. A method of intubation catheter placement, comprising:
inserting a distal end of a tube changer device into the proximal end of the lumen of the first intubation catheter, the tube changer device comprising:
a tubular shaft having a hollow interior forming a main lumen extending from a proximal end to a distal end; and
a dilator portion at the distal end, the dilator portion having a tapered profile with an outer diameter that varies along a dilator length measured from a junction point on the tubular shaft to a blunt tip, the main lumen extending through the dilator portion to the blunt tip;
advancing the tube changer device through the lumen of the first intubation catheter until the blunt tip of the dilator portion of the tube changer device is positioned substantially aligned with the distal end of the first intubation catheter;
holding the tube changer device in position;
removing the first intubation catheter by sliding the first intubation catheter over the tube changer device;
placing a distal end of a second intubation catheter around the proximal end of the tube changer device;
advancing the second intubation catheter guided by the tube changer device until a distal portion of the second intubation catheter surrounds the dilator portion of the tube changer device;
holding the second intubation catheter in position; and
removing the tube changer device from the second intubation catheter.

15. The method of claim 14, further comprising inserting a stylet into the lumen of the first intubation catheter to use a guide, wherein:
inserting a distal end of the tube changer device into the proximal end of the lumen of the first intubation catheter comprises threading the tube changer device over the stylet, and
removing the tube changer device from the second intubation catheter comprises removing the stylet with the tube changer device.

16. A method of intubation catheter placement, comprising:
positioning a tube changer device within a lumen of a replacement intubation catheter, the tube changer device comprising:
a tubular shaft having a hollow interior forming a main lumen extending from a proximal end to a distal end; and
a dilator portion at the distal end, the dilator portion having a tapered profile with an outer diameter that varies along a dilator length measured from a junction point on the tubular shaft to a blunt tip, the main lumen extending through the dilator portion to the blunt tip;
wherein the tube changer device is configured to be slidably received within the replacement intubation catheter and the blunt tip of the dilator portion of the tube changer device is positioned substantially aligned with the distal end of the replacement intubation catheter;
advancing, together, the tube changer device and the replacement intubation catheter until the blunt tip of the tube changer device reaches a position;
holding the replacement intubation catheter in position; and
removing the tube changer device from the replacement intubation catheter.

17. The method of claim 16, further comprising:
inserting a stylet into the lumen of the existing intubation catheter to use a guide;
advancing, together, the tube changer device and the intubation catheter guided by the stylet until the blunt tip of the tube changer device reaches a position;
holding the replacement intubation catheter in position; and
removing the tube changer device and stylet by sliding the tube changer device through the lumen of the replacement intubation catheter.
